(19) United States
(12) Patent Application Publication
(10) Pub. No.: US 2006/0121006 A1
(43) Pub. Date: Jun. 8, 2006

(54) Production of nutritional and therapeutic products from cultured animal cells

(76) Inventors: Michael B. Chancellor, Pittsburgh, PA (US); Brandon S. Minnery, Stafford, VA (US); David D. Chancellor, Pittsburgh, PA (US)

Correspondence Address: THE WEBB LAW FIRM, P.C., 700 KOPPERS BUILDING, 436 SEVENTH AVENUE, PITTSBURGH, PA, 15219, US

(21) Appl. No.: 11/224,676

(22) Filed: Sep. 12, 2005

Related U.S. Application Data
(60) Provisional application No. 60/608,676, filed on Sep. 10, 2004.

(51) Int. Cl. A61K 35/14; A61K 35/12; C12N 5/06

(52) U.S. Cl. 424/93.7; 435/325

(57) ABSTRACT

The present invention describes methods and products involving the use of cultured animal cells as ingredients in nutritional and therapeutic products. Said animal cells are preferably cultured on an industrial scale prior to their incorporation into any of a number of edible, topical, oral, or other products.

### CROSS REFERENCE TO RELATED APPLICATIONS

This application claims the benefit of U.S. Provisional Patent Application No. 60/608,676 filed Sep. 10, 2004, which is hereby incorporated by reference in its entirety.

### FIELD OF THE INVENTION

The present invention generally relates to the use of cultured animal cells for the production of nutritional, medicinal, or therapeutic products, particularly wherein said cells are derived from rare or endangered species. Also included are edible meat-containing products suitable for consumption by humans and other organisms, whether as food or as nutritional supplements. The invention also relates to the use of large-scale culturing methods for proliferating the cells prior to their incorporation into said products. The invention encompasses the methods of manufacturing the products, the products themselves, and methods of using the products.

### BACKGROUND OF THE INVENTION

The use of animal tissues by humans—whether for food, for medicinal purposes, or for religious or cultural purposes—presents a number of ethical, environmental and public health dilemmas. Here we disclose methods of using cultured animal cells, either alone or in combination with other ingredients, as components of nutritional and/or therapeutic end products. Also disclosed are the products themselves, each of which contains as an essential ingredient an end population of animal cells derived from in vitro cultures.

In one embodiment of the present invention, animal cells (including but not limited to muscle-derived cells, hereafter termed MDCs) are collected from a donor animal, expanded in vitro, and included in a food or nutritional product suitable for consumption by humans and/or other organisms. Given the controlled conditions of in vitro cell culturing methods, and given that large cell populations can be derived from a single, disease-free animal, this embodiment of the invention provides an attractive alternative to conventional meat products and their attendant health risks. For example, the recent identification of “mad cow” disease (bovine spongiform encephalopathy) has highlighted the vulnerability of both human and animal populations to the contamination of our animal food supply by infectious agents—whether through natural causes or through an act of bioterrorism. Furthermore, the animal tissue explants necessary to begin the cell culture process can be obtained in a painless procedure which does not require the slaughter of the donor animal, thereby circumventing many of the ethical and environmental issues associated with the use of animals for food. Yet another benefit is that the invention makes possible the consumption of cells and tissues from rare or endangered animal species without further depleting the ranks of such species.

The market potential for this embodiment of the invention is extremely promising for several reasons. First, livestock populations used as food sources are continuously threatened by diseases which may in some circumstances spread to humans. Because animal flesh is widely consumed in many societies, large portions of the population are susceptible to infectious agents or pathogens spread through tainted meat, such as prion diseases (e.g. bovine spongiform encephalopathy) and viruses (e.g. avian influenza). Any such epidemic, whether induced by natural causes or by a deliberate act of bioterrorism, has the potential to ramify throughout the food chain causing devastation to both human and animal populations. The present invention provides a safety net against this type of scenario by substituting animal tissue cultivated under controlled conditions in place of conventional meat sources. For instance, the original tissue explants from which subsequent cultures are grown can be harvested from a small population of animals raised in a well-regulated, disease-free environment. In addition, once cell populations are proliferating in vitro, they can be further monitored under controlled conditions and assayed repeatedly for the presence of toxins and infectious agents using methods known in the art.

Yet another promising feature of this embodiment of the invention is that select phenotypes of animal cells can be grown in culture, allowing for a healthier and more delectable food product. Conventional meat products may contain fat, connective tissue, dermal tissue, etc., in addition to lean muscle tissue. By choosing appropriate culturing conditions, however, it is possible to selectively isolate and grow specific cell populations. Thus, a final product low in saturated fat could be produced without requiring the removal of offal, tendons, etc. Considering the prevalence of cardiovascular disease in developed countries, in particular the United States, in vitro animal cells could provide much healthier alternatives to most commercial meat products.

In another embodiment of the invention, cultured animal cells—including tissues, organs, secretions, and bioproducts derived thereof—are used as ingredients in medicinal or therapeutic products, preferably as substitutes for cells or tissues harvested from rare, exotic, or endangered animals. For instance, the use of tiger and rhinoceros tissues in Traditional Chinese Medicine (TCM) and other non-western medical practices has threatened the survival of these species, as increasing numbers of animals are killed for their body parts, including fluids such as blood, secretions such as musk and bile, tissues and organs such as bone and teeth, and other bioproducts (Ellis, 2005, Tiger Bone & Rhino Horn: The Destruction of Wildlife for Traditional Chinese Medicine). Animal-derived components continue to feature prominently in the pharmacopeia of TCM, and the market for products that contain such components is thriving. Examples of products and their uses include tiger bone used as an anti-inflammatory drug to treat rheumatism and arthritis, tiger bile used to treat convulsions associated with meningitis, tiger fat used to treat leprosy and rheumatism, tiger brain used to treat laziness and pimples, tiger penis used as an aphrodisiac, tiger teeth used to treat fever, tiger claws used as a sedative for insomnia, tiger liver used to impart courage, musk from deer of the genus Moschus used as an aphrodisiac and as a treatment for acute pain and swelling of the abdomen, elephant penis used as an aphrodisiac, bear bile used to treat inflammation and pain, rhinoceros horn used to treat a variety of ailments, rhinoceros liver used to cure tuberculosis, and pangolin scales used to to treat skin infections, to promote blood circulation and to stimulate milk secretion. By embracing the use of in vitro cell culture and tissue engineering methods for creating substitutes for animal-derived components, the present embodiment envisions an entirely new class of medicines, therapeutics, pharmaceuticals, cosmetics and the like which incorporate animal-derived components but which do not inflict significant animal suffering and which do not threaten rare or endangered species.

A generally attractive aspect of this invention is that under most contemplated embodiments it allows for the creation of products containing animal components without necessitating the slaughter of animals. Many individuals abstain from eating meat on the ethical grounds that the meat-processing industries inflict large-scale suffering on animal populations. Similarly, many people avoid using any product containing animal parts for the same reason, namely that harvesting the parts of animals causes suffering and may also result in damage to the environment. Although the in vitro cell cultures described in the present invention may be derived from living donor animals, the biopsy procedure for obtaining a tissue sample, e.g. a muscle biopsy, can be performed under local anesthesia with little discomfort to the animal. Moreover, the use of a small population of donor animals ultimately places less strain on environmental resources. For example, cattle destined for the beef industry normally require substantial territory for grazing—land which could otherwise be put to more efficient agricultural use.

### DETAILED DESCRIPTION OF THE INVENTION

The present invention provides for the use of cultured animal cells for use in nutritional and therapeutic products. One embodiment encompasses the use cultured animal cells, preferably muscle-derived cells (MDCs), as a source of food or nutritional supplement for humans and/or other organisms. A method for obtaining MDCs is described herein as well as in co-pending applications, U.S. Ser. Nos. 09/302,896 and 09/549,937, the contents of which are herein incorporated by reference in their entireties. Also described herein are methods for the large-scale culturing of animal cells and the incorporation of said cells into edible end products, either alone or in combination with other ingredients. Additional disclosure is directed towards a method of altering or enhancing in vivo animal tissues through the introduction of cultured animal cells, wherein the cultured cells are integrated into the host tissue and the resulting tissue is harvested for consumption. Another related embodiment concerns the use of cultured animal cells, tissues, organs, bioproducts and the like in medicinal and/or therapeutic products, preferably as substitutes for native cells, tissues, organs, bioproducts and the like.

The present invention provides for the use of cultured animal cells, i.e. animal cells grown in vitro, as a source of food. The term animal cells encompasses cells derived from any and all animal tissues, including, but not limited to, skeletal muscle tissue, smooth muscle tissue, connective tissue, neuronal tissue, fat tissue, bone tissue, and any combination thereof. Also included as animal cells are cells in various stages of lineage commitment, or differentiation, such as stem cells, satellite cells and other precursor cell phenotypes. The present invention also contemplates the use of commercially available cell lines, transformed cell populations, clonal cell populations, and any cell populations derived from primary biopsies or explants.

Preferably, the animal cells used in the present invention are muscle-derived cells (MDCs). MDCs can be isolated from primary skeletal muscle explants as an enriched end population of early muscle-derived cells, such as myoblasts, by a series of plating and culturing steps. The culturing enrichment results in an end population of MDCs which are viable, non-fibroblastic, desmin-positive cells that can form muscle fibers (myofibers). During the successive platings of cells into new containers, MDCs (including myoblasts and muscle-derived early cells) are enriched and separated from non-MDC types, e.g., fibroblasts and adipocytes, endothelial cells, and connective tissue cells. The successive platings can be carried out for about 3-7 days, or 4-6 days, or 3-5 days, until non-fibroblast MDCs remain and proliferate in the cultures. During the culturing period, the adherent fibroblasts and non-muscle cells are essentially depleted from successive cell cultures by passaging the cell suspension of muscle cells into new tissue culture dishes or flasks, which may or may not be collagen-coated. In the successive platings, adherent fibroblasts are removed. The muscle cells are successively passaged into new tissue culture dishes or flasks with new culture medium, e.g., at about 24 hour intervals, until the MDCs remain enriched in the cultures as viable and proliferating cells, with very little fibroblastic cell component, for example, at the end of a series of 3-7 platings, 4-6 platings, or 3-5 platings. The end population of MDCs is enriched for cells that can form muscle fibers, e.g., myoblast cells or early muscle-derived cells, when injected into animal tissue, e.g., muscle tissue, in which the MDCs survive, repopulate and proliferate. MDC populations isolated in accordance with the above methods also exhibit sustained viability and replicative capacity in culture conditions, i.e. in vitro, and therefore are ideally suited to be grown on an industrial scale for use as food. It should also be noted that variations of the above procedure which effectively isolate MDCs from other cellular phenotypes based on differential adherence characteristics are also encompassed by the present invention. For example, cell populations need not necessarily be transferred from one discrete container to another; rather, a rotating cylindrical flask with variable rotational speed may be used instead. In this case, different cell populations will adhere during different phases of the rotational cycle and will therefore come to occupy distinct areas of the flask's inner surface. Likewise, a starting population of cells may be slowly passaged through a tube, pipe, column, or similar device in which a matrix comprised of collagen, polystyrene, or other elements to which cells adhere, is used to sort the cells based on differential adherence properties. This latter method is analogous to the use of an affinity column to purify proteins. MDCs may also be isolated using other techniques commonly known in the art, including, but not limited to, fluorescence-activated cell sorting (FACS).

In all aspects of the present invention, it is preferred that large-scale (i.e. industrial-scale) cell culturing methods be used to grow the animal cells prior to their incorporation into edible end products. Examples of large-scale culturing systems, or bioreactors, include any incubator containing multiple culture flasks having surface areas preferably greater than 75 cm², any system consisting of stackable growth chambers wherein the chambers are arranged in parallel either vertically or horizontally, and any system which utilizes roller bottle flasks, preferably wherein the flask surfaces contain pleats, folds, ridges, or other patterns which maximize growth surface. Also included are spinner flasks, stirred tanks reactors, and disposable and/or reusable bag bioreactors, preferably of a volume greater than 10 liters and more preferably of a volume greater than 200 liters. Further included are systems specially adapted for the culturing of anchorage dependent cells, particularly those that utilize microcarrier technology either in a packed-bed or fluidized-bed reactor system. Microcarriers may comprise any of a number of structures, e.g. spheres, beads, hexagons, sheets, fibrous strands, fixed matrices and the like, and may comprise various elements alone or in combination, including—but not limited to—collagen, chitin, polystyrene, polyester and polypropylene. In those embodiments where the microcarriers are to be included in the end product, it is preferred that the constituent material(s) be non-toxic, e.g. collagen. In order to optimize cell growth conditions and cell viability, it is further preferred that any large-scale culture system employed in the practice of the present invention be of sufficient technical sophistication that parameters such as temperature, agitation, pH, dissolved O₂, and the like may be placed under automated control.

In one aspect of the present invention, MDCs are injected, administered or transplanted either systemically or locally into an animal, are allowed a period of time in which to proliferate and integrate with host tissue, and are then harvested along with surrounding tissues for use as food. The MDCs confer unique textural, gustatory and biochemical properties that are not otherwise present in the native, or wild type, meat. MDCs according to this particular aspect of the invention may be derived from a donor animal biopsy, preferably a skeletal muscle biopsy, and may be isolated and expanded according to techniques known in the art, including, but not limited to, those techniques described in co-pending applications, U.S. Ser. Nos. 09/302,896 and 09/549,937, the contents of which are herein incorporated by reference in their entireties. Also in accordance with this aspect of the invention, the MDC's may be optionally engineered to express, under-express, or over-express one or more heterologous or endogenous polypeptides, proteins or gene products. In the case of under-expression, it is preferred that the gene which encodes the protein myostatin be silenced or suppressed, as recent studies indicate that reduced expression of this protein allows for increased muscle growth and proliferation. MDCs according to this aspect may be autologous, allogeneic, or xenogeneic to the recipient animal. Xeneogeneic injections may be desirable to produce novel hybrid meats including but not limited to: pork/beef, beef/shrimp (“surf and turf”), salmon/shark, and rattlesnake/koala. Moreover, hybrid meats may contain tissues from more than two animal species.

Regarding xenogeneic MDC injections, our lab has previously demonstrated that mouse MDCs injected into rats can survive and integrate into host muscle tissue. Preliminary experiments also show that human MDCs can survive for at least 7 days when injected into immunodeficient rats (disclaimer: the inventors do not advocate the consumption of human meat or human cells, though such practice does fall within the scope of the current invention). The viability of allogeneic and xenogeneic MDCs may in part relate to the presence of stem cells within the MDC population, as stem cells have been shown to exhibit immune privilege under certain scenarios (Cannon, T. W., Lee, J. Y., Somogyi, G., Pruchnic, R., Smith, C. P. Huard, J., Chancellor, M. B.: Improved sphincter contractility after allergenic muscle-derived progenitor cell injection into the denervated rat urethra. Urology; 62:958-963, 2003). However, in order to further promote the survival of injected cells, it is alternatively possible to employ any of a number of immunosuppressive therapies known in the art. Recently, for example, a transgenic pig model has been developed that allows for immunocompatible xenogeneic transplant of porcine tissues into other species. These pigs lack one allele of the α1,3-galactosyltransferace gene. MDCs harvested from these transgenic pigs are particularly amenable to injection into other species. Accordingly, one embodiment of this aspect of the invention involves the use of MDCs harvested from said transgenic pigs to create hybrid meats according to the above methods.

In another aspect of the invention, MDCs are grown in a monolayer either on a standard culture surface (e.g. polystyrene) or on any of a number of tissue engineering scaffolds and matrices known in the art. The resulting monolayer may then be consumed as a whole, or depending on its size, may be sectioned into smaller strips or other shapes more amenable to consumption. At any point before or after the sectioning of the monolayer, the MDCs may be cooked, baked, smoked, dehydrated, marinated, basted, seasoned, or enhanced with flavor supplements and/or other additives and preservatives. Additionally, at any point prior to consumption, the MDC-containing monolayers/strips/pieces may be further subjected to mechanical forces which alter the shape, consistency, and/or textural properties of the product. In the preferred embodiment of this aspect, the MDC-containing monolayers/strips/pieces are cultured under conditions (e.g. mechanical stress and/or strain, including intermittently applied stress and/or strain) which result in a texture that is mimetic of native muscle tissue. As a further variation of the above process, the monolayer may either be combined with other layers of cultured animal cells, or may be rolled, stacked, folded, twisted, or crumpled up upon itself to add 3-dimensionality to the structure. The resulting product can be further cut, sectioned, or ground into smaller pieces suitable for consumption. NOTE: The matrix upon which the monolayer is grown is an optional component. The matrix, if used, may remain as an intact component of the edible end product, or alternatively may be composed of biosorbant components (e.g. polylactic acid or polyglycolic acid) such that the matrix degrades either partially or completely prior to consumption. NOTE: The MDCs used in this aspect of the invention may be further engineered to express, under express, or over-express any number of amino acids, peptides proteins, and gene products. Also, MDCs or other cellular phenotypes (e.g. fibroblasts and/or adipocytes) from multiple species may be seeded onto the same monolayer. Additionally, different monolayers containing MDCs from different species may be alternately stacked, rolled, folded, crumpled, or otherwise combined into an integrated end product. The initial monolayer structure, or indeed any layered or stratified structure, need not be preserved or evident in the final product.

In another aspect of the invention, MDCs are cultured on microcarrier beads in suspension as described in paragraph 0011 of the present application. The MDCs are dissociated from the collagen beads using techniques known in the art and are collected for further processing, e.g. flavoring, in preparation for their incorporation into an edible end product. Alternatively, the MDCs may be further seeded onto additional matrices and/or carriers for additional growth and manipulation prior to being incorporated into an edible end product. In another variation of this aspect, the MDCs are allowed to remain adherent to the microcarrier, and the MDC-coated beads are themselves used as a component in an edible end product. The beads will provide additional mass and roughage to the food product. If included as part of the food product along with the MDCs, the microcarrier beads will preferably be composed of nontoxic materials. The MDC/beads may be compressed-into a solid substance and introduced as a component of solid food products, or may be consumed in suspension as part of a slurry or other liquid food product.

Also contemplated by the present invention is the use of MDC-seeded casings for sausages, vegetarian sausages, and/or other processed food products. According to this aspect of the invention, MDCs are seeded onto a matrix and the resulting construct is used as a casing for sausage or a related product. Commercially available sausage casings include collagen casings, regenerated cellulose casings, and natural casings comprised of intestinal submucosa. Two of these materials, collagen and intestinal submucosa (which itself contains collagen as a principal component), have already been shown to function as excellent scaffolds for the growth and proliferation of animal cells in tissue engineering applications. MDC-seeded casings can be further built up from multiple layers of seeded matrices in order to add thickness and integrity to the casing. The MDC-containing casings can be filled with a variety of edible products including, but not limited to, conventional meat or poultry-based sausage material, vegetarian sausage material, and sausage material containing animal cells grown in vitro and processed in accordance with any of the aforementioned methods and descriptions.

In all aspects of the present invention, the edible end products include, but are not limited to, patties, sausages, sausage casings, hot dogs, purees, pastes, gels, slurries, stews, soups, broths, baby foods, jerkies, snack sticks, crumbles, pellets, wafers, chips, powders, animal feed, and the like. The end products each contain some portion of cultured animal cells as an essential ingredient but may also contain plant-derived matter (including cultured plant cells), fungal matter, and/or other non-absorbable, non-toxic substances. The present invention also envisions the incorporation of MDCs into standard commercial vegetarian and/or meatless products, the ingredients of which may comprise one or more of the following: water, soy protein concentrate, wheat gluten, soy sauce, chicory fiber, evaporated cane juice, salt, malt extract, wheat starch.

The MDCs and/or other animal cell phenotypes embraced by the present invention may be engineered ex vivo, e.g. in vitro, to express, over-express, or under-express one or more gene products, whether native (wild type) or exogenous (foreign), using any combination of techniques commonly known in the art. Non-limiting examples of gene products include proteins, polypeptides, peptides, hormones, metabolites, growth factors, drugs, enzymes, and the like. In the case where expression or over-expression is desired, the gene product(s) will be ones which confer unique nutritional, gustatory, medicinal or salutary properties to the cells, or which otherwise enhance or regulate the growth, differentiation, or division of cells in vitro prior to their processing for consumption. A non-limiting example of the above is the engineering of cells to express specific viral antigens which, upon consumption, function to vaccinate the consumer against a particular viral infection. One non-limiting example of a case in which under-expression is desired is that of the protein myostatin, the suppression of which has been shown to result in increased growth of muscle cells. A non-limiting example of a gene whose expression or over-expression is desired is the gene for telomerase.

The animal cells, including MDCs, can be genetically engineered by a variety of molecular techniques and methods known to those having skill in the art, for example, transfection, infection, transduction, or direct DNA injection. Transduction as used herein commonly refers to cells that have been genetically engineered to contain a foreign or heterologous gene via the introduction of a viral or non-viral vector into the cells. Viral vectors are preferred. Transfection more commonly refers to cells that have been genetically engineered to contain a foreign gene harbored in a plasmid, or non-viral vector. Animal cells can be transfected or transduced by different vectors and thus can serve as gene delivery vehicles to allow the gene products to be expressed and produced at and around the tissue or organ site.

Although viral vectors are preferred, those having skill in the art will appreciate that the genetic engineering of cells to contain nucleic acid sequences encoding desired proteins or polypeptides, cytokines, and the like, may be carried out by methods known in the art, for example, as described in U.S. Pat. No. 5,538,722, including fusion, transfection, lipofection mediated by precipitation with DEAE-Dextran or calcium phosphate (Graham and Van Der Eb, 1973, Virology, 52:456-467; Chen and Okayama, 1987, Mol. Cell. Biol. 7:2745-2752; Rippe et al., 1990, Mol. Cell. Biol., 10:689-695); gene bombardment using high velocity microprojectiles (Yang et al., 1990, Proc. Natl. Acad. Sci. USA, 87:9568-9572); microinjection (Harland and Weintraub, 1985, J. Cell Biol., 101:1094-1099); electroporation (Tur-Kaspa et al., 1986, Mol. Cell. Biol., 6:716-718; Potter et al., 1984, Proc. Natl. Acad. Sci. USA, 81:7161-7165); DNA (vector)-loaded liposomes (Fraley et al., 1979, Proc. Natl. Acad. Sci. USA, 76:3348-3352); lipofectamine-DNA complexes; cell sonication (Fechheimer et al., 1987, Proc. Natl. Acad. Sci. USA, 84:8463-8467); receptor-mediated transfection (Wu and Wu, 1987, J. Biol. Chem., 262:4429-4432; Wu and Wu, 1988, Biochemistry, 27:887-892); and the like. In one alternative, the retroviral or plasmid vector can be encapsulated into a liposome, or coupled to a lipid, and then introduced into a cell. In addition, cDNA, synthetically produced DNA, or chromosomal DNA can be employed as vector inserts utilizing methods and protocols known and practiced by those having skill in the art.

Standard protocols for producing replication-deficient retroviruses, including the steps of 1) incorporating exogenous genetic material into a plasmid; 2) transfecting a packaging cell line with plasmid and production of recombinant retroviruses by the packaging cell line; 3) collecting viral particles from tissue culture media; and 4) infecting the target cells with viral particles, are provided in, e.g., M. Kriegler, 1990, “Gene Transfer and Expression, A Laboratory Manual,” W. H. Freeman Co., NY; and E. J. Murry, Ed., 1991, “Methods in Molecular Biology,” vol. 7, Humana Press, Inc., Clifton, N. J.

Expression vectors containing all the necessary elements for expression are commercially available and known to those skilled in the art. See, e.g., Sambrook et al., 1989, Molecular Cloning: A Laboratory Manual, Second Edition, Cold Spring Harbor Laboratory Press, Cold Spring Harbor, N.Y.; F. M. Ausubel et al. (eds), 1995, Current Protocols in Molecular Biology, John Wiley & Sons, Inc., New York, N.Y.; D. N. Glover (ed), 1985, DNA Cloning: A Practical Approach, Volumes I and II; M. L. Gait (ed), 1984, Oligonucleotide Synthesis; Hames and Higgins (eds), 1985, Nucleic Acid Hybridization; Hames and Higgins (eds), 1984, Transcription and Translation; R. I. Freshney (ed), 1986, Animal Cell Culture; Immobilized Cells and Enzymes, 1986, (IRL Press); Perbal, 1984, A Practical Guide to Molecular Cloning; The Series, Methods in Enzymology, Academic Press, Inc.; J. H. Miller and M. P. Calos (eds), 1987, Gene Transfer Vectors for Mammalian Cells, Cold Spring Harbor Laboratory; Wu and Grossman (eds), Methods in Enzymology, Vol. 154; Wu (ed), Methods in Enzymology, Vol. 155.

Illustrative examples of vehicles or vector constructs for transfection or infection of animal cells, including MDCs, according to the present invention include replication-defective viral vectors, DNA virus or RNA virus (retrovirus) vectors, such as adenovirus, herpes simplex virus and adeno-associated viral vectors. Preferred are adenovirus vectors. Such vectors include one or more promoters for expressing a heterologous molecule, e.g., a bioactive molecule (e.g., protein, polypeptide, or peptide). Suitable promoters which can be employed include, but are not limited to, adenoviral promoters, such as the adenoviral major late promoter; heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex Virus thymidine kinase promoter; retroviral LTRs (including modified retroviral LTRs); the β-actin promoter; and human growth hormone promoters. The promoter also may be the native promoter that controls the nucleic acid sequence encoding the polypeptide. Preferred viral vectors are typically derived from non-cytopathic eukaryotic viruses in which non-essential genes have been replaced with the nucleic acid sequence(s) of interest. Non-cytopathic viruses include retroviruses, which replicate by reverse transcription of genomic viral RNA into DNA with subsequent proviral integration into host cellular DNA.

Retroviruses have been approved for human gene therapy trials. In general, the retroviruses are replication-deficient, i.e., capable of directing synthesis of the desired proteins, but incapable of manufacturing an infectious particle. Retroviruses from which the retroviral plasmid vectors can be derived include, but are not limited to, Moloney murine leukemia virus, spleen necrosis virus, retroviruses such as Rous sarcoma virus, Harvey sarcoma virus, avian leukosis virus, gibbon ape leukemia virus, human immunodeficiency virus, adenovirus, myeloproliferative sarcoma virus, and mammary tumor virus. In general, the retroviruses used to create a viral vector are preferably debilitated or mutated in some respect to prevent disease transmission. If desired, infectious replication-defective viral vectors may be used to genetically engineer the cells prior to in vivo injection of the cells. In this regard, the vectors may be introduced into retroviral producer cells for amphotrophic packaging. The natural expansion of muscle-derived progenitor cells into adjacent regions obviates a large number of injections into or at the site(s) of interest.

The vectors are typically substantially free of any prokaryotic DNA and may comprise a number of different functional nucleic acid sequences. Examples of such functional sequences include nucleic acid, e.g., DNA or RNA, sequences comprising transcriptional and translational initiation and termination regulatory sequences, including promoters (e.g., strong promoters, inducible promoters, and the like) and enhancers, which are active, for example, in esophagus or small intestine cells. Also included as part of the functional sequences is an open reading frame (nucleic acid sequence) encoding a protein, polypeptide, or peptide of interest. Flanking sequences may also be included for site-directed integration. In some situations, the 5′-flanking sequence allows for homologous recombination, thus changing the nature of the transcriptional initiation region, so as to provide for inducible or noninducible transcription to increase or decrease the level of transcription, as an example.

The vector employed generally also includes an origin of replication and other genes that are necessary for replication in the host cells, as routinely employed by those having skill in the art. As an example, the replication system comprising the origin of replication and any proteins associated with replication encoded by a particular virus may be included as part of the construct. The replication system must be selected so that the genes encoding products necessary for replication do not ultimately transform the animal cells themselves when transformation is not desired. Such replication systems are represented by replication-defective adenoviruses constructed as described, for example, by G. Acsadi et al., 1994, Hum. Mol. Genet. 3:579-584, and by Epstein-Barr virus. Examples of replication defective vectors, particularly retroviral vectors that are replication defective, are BAG, described by Price et al., 1987, Proc. Natl. Acad. Sci. USA, 84:156; and Sanes et al., 1986, EMBO J., 5:3133. It will be understood that the final gene construct can contain one or more genes of interest, for example, a gene encoding a bioactive metabolic molecule.

In general, the nucleic acid sequence desired to be expressed by the animal cells is that of a structural gene, or a functional fragment, segment or portion of the gene, which is heterologous to the cell serving as delivery vehicle and which encodes a desired protein or polypeptide product. The encoded and expressed product may be intracellular, i.e., retained in the cytoplasm, nucleus, or an organelle of a cell, or may be secreted by the cell. For secretion, the natural signal sequence present in the structural gene may be retained, or a signal sequence that is not naturally present in the structural gene may be used. When the polypeptide or peptide is a fragment of a protein that is larger, a signal sequence may be provided so that, upon secretion and processing at the processing site, the desired protein will have the natural sequence. Examples of genes of interest for use in accordance with the present invention include genes encoding cell growth factors, suppressor molecules, cell differentiation factors, cell signaling factors and programmed cell death factors. Specific examples include, but are not limited to, genes encoding BMP-2 (rhBMP-2), IL-1Ra, Factor IX, and connexin 43.

A marker may optionally be used for the selection of cells containing the vector construct. The marker may be an inducible or non-inducible gene and will generally allow for positive selection under induction, or without induction, respectively. Examples of commonly used marker genes include neomycin, dihydrofolate reductase, glutamine synthetase, and the like. The vector employed also generally includes an origin of replication and other genes that are necessary for replication in the host cells, as routinely employed by those having skill in the art. As an example, the replication system comprising the origin of replication and any proteins associated with replication encoded by a particular virus may be included as part of the construct.

Another embodiment of the present invention pertains to the use of cultured animal cells—and/or any tissues, organs, parts, secretions, or bioproducts derived thereof—as components of medicinal and/or therapeutic products. The term therapeutic here refers to any product whose ingestion, consumption, absorption, injection, inhalation, application, etc., causes the amelioration or lessening of any physical, psychological, social or spiritual illness, problem, deficit and the like. In the preferred version of this embodiment, the animal cells are derived from rare, exotic, extinct, or endangered species. The term endangered species here refers to any species contained in the World Conservation Union's (IUCN) Red List of Endangered Species. Examples of species contemplated for use in this and other embodiments of the current invention include any and all species, whether or not endangered, of: rhinoceros, tiger, bear, musk deer, turtle, tortoise, snake, bird, elephant, seal, walrus, whale, shark, pig, gorilla, chimpanzee. The preceding are intended as non-limiting examples of species.

Cells types contemplated for use in this embodiment include any and all cells capable of being cultivated and/or proliferated in vitro. Preferred are stem cells, including but not limited to embryonic stem cells, adult stem cells, totipotent stem cells, pluripotent stem cells, multipotent stem cells, and precursor cells. Further examples of preferred stem cell types include adult neuronal progenitor cells, neural stem cells, multipotent stem cells from subventricular forebrain region, ependymal-derived neural stem cells, hematopoetic stem cells, liver-derived hematopoetic stem cells, marrow-derived stem cells, adipo-fibroblasts, adipose-derived stem cells, islet-producing stem cells, pancreatic-derived pluripotent islet-producing stem cells, mesenchymal stem cells, bone marrow stromal cells, muscle side population cells, muscle main population cells, skeletal muscle satellite cells, muscle-derived stem cells, bone marrow-derived recycling cells, blood-derived mesenchymal precursor cells, bone-marrow derived side population cells, muscle precursor cells, circulating skeletal stem cells, neural progenitor cells, multipotent adult progenitor cells, mesodermal progenitor cells, spinal cord progenitor cells, and spore-like cells (REF, Young and Black, 2004). Additional preferred cell types include myoblasts, osteoblasts, fibroblasts, lipoblasts, and odontoblasts, as well as the MDCs described in preceding paragraphs.

The tissues, organs or body parts encompassed by this aspect of the invention include, without limitation: skin, bones, teeth, tusks, blood, fat, cartilage, central nervous tissue, peripheral nervous tissue, horns, antlers, claws, nails, scales, talons, tongue, liver, eye, penis, testicles, scrotum, hair, fur, whisker, gall bladder, intestine, male and female genitourinary tissue, and connective tissue. Non-limiting examples of secreted materials and bioproducts include musk and bile.

Non-limiting examples of medicinal and/or therapeutic products include the following: pills, capsules, tablets, gums, powders, gelatins, cubes, teas, poultices, granules, chips, flakes, grinds, wafers, creams, pastes, ointments, balms, plasters, soaps, gels, shampoos, salves, rubs, lotions, liquids, injectable solutions, suppositories, wines, juices, smokeable agents, incense sticks, inhalants, potions, tonics, elixirs, talismans, amulets, tokens, charms, and jewelry. Also included are the edible end products listed previously—including patties, sausages, sausage casings, hot dogs, purees, pastes, gels, slurries, stews, soups, broths, baby foods, jerkies, snack sticks, crumbles, pellets, wafers, chips, powders, animal feed, and the like—wherein said end products contain as a therapeutic/medicinal ingredient an animal-derived components, such as cells, tissues, organs and the like.

The present invention specifically provides for a method of producing medicinal/therapeutic product having as an ingredient tiger bone or tiger bone cells. Describe incorporation of osteoblasts, mesenchymal stem cells, etc, into pills, wine, etc.

Additional types of cells may be used in the present invention, including but not limited to tooth cells (odontoblasts), tissue-engineered penis, creams containing cultured animal fat cells, compositions containing musk secretions, bile secretions and other secretions.

The invention claimed is:

1. A method for producing nutritional and/or therapeutic cells in vitro, comprising:
selecting a quantity of cells for cell culture, wherein said cells are, predominantly mammalian;
seeding said cells onto a carrier;
growing the cells into a monolayer; and
harvesting the monolayer for use in the preparation of an edible, consumable substance for use as a nutritional and/or therapeutic product.

2. The method according to claim 1, wherein said carrier is a bead.

3. The method according to claim 1 wherein said carrier is a microbead.

4. The method according to claim 1 wherein said carrier is a sheet.

5. The method according to claim 1 wherein said carrier is a nontoxic polymer.

6. The method according to claim 1 wherein said carrier is selected from the group consisting of a collagen-containing substance, a chitin-containing substance, a polylactic acid polymer and a polyglycolic acid polymer.

7. The method according to claim 6 wherein said carrier is a collagenous sheet.

8. The method according to claim 7 wherein said cells are deposited onto said collagenous sheet prior to growing the cells into a monolayer.

9. The method according to claim 8 wherein said monolayer is used to prepare an edible or consumable product by any or all of the steps of starking, rolling, folding, crumpling, or mixing of the monolayer to make either a solid construct or individual cells for further incorporation in a nutritional and/or therapeutic composition.

10. The method according to claim 9 wherein the mammalian cells are all from a single animal.

11. The method according to claim 10 wherein the mammalian cells are all autologous to a single animal and said animal is selected from the group consisting of rhinoceros, tiger, bear, musk deer, turtle, tortoise, snake, bird, elephant, seal, walrus, whale, shark, pig, gorilla, chimpanzee, deer, and pangolin.

12. The method according to claim 10 wherein the cells are from two animal or plant species.

13. The method according to claim 12 wherein said two animal or plant species give hybrid heterogeneous mixtures of cells.

14. The method according to claim 12 wherein one of said species is mammalian and one of said species is non-mammalian.

15. The method according to claim 14 wherein said non-mammalian species is selected from a plant or a fungus.

16. The method according to claim 14 wherein at least one of said species has cells which have been genetically engineered.

17. The method according to claim 16 wherein the cells which have been genetically engineered are cells from a transgenic animal which already contains immunocompatible xenogeneic cells.

18. The method according to claim 6 wherein said carrier is a bead comprised of any nutritive fiber.

19. The method of claim 1 wherein, in the preparation of the edible substance, the cells in the monolayer are further treated.

20. The method of claim 1 wherein the cells are not all muscle cells.

21. The method according to claim 9 wherein the edible product is prepared by removing individual or pluralities of cells from the carrier and incorporating them into a nutritional or therapeutic composition.

22. A nutritional and/or therapeutic product comprising: an edible quantity of cells harvested and grown from at least one animal and predominantly further comprising mammalian cells, characterized in that the growing of said edible quantity of cells is accomplished on a carrier.

23. The nutritional and/or therapeutic product according to claim 22 wherein said carrier is selected from the group consisting of a bead and a sheet which further comprise acellular cadaveric skin matrix, poly-lactic acid, hyaluronic acid, polyglycolic acid, polyethylene oxide, polybutylene terephthalate, silicone, de-epidermized dermis, collagen sponge, collagen-chitosan sponge, chitosan-cross-linked collagen-glycosaminoglycan matrix, collagen gel, polygalactin mesh and small intestinal submucosum.

24. The nutritional and/or therapeutic product according to claim 22 wherein said carrier is a collagenous sheet and suitable for xenogeneic implantation.

25. The nutritional and/or therapeutic product according to claim 24 wherein the product including the collagenous sheet is further processed into a supplement.

26. The nutritional and/or therapeutic product according to claim 15 wherein a quantity of xenogeneic cells are also present, to create a hybrid product containing tissues from more than two species.

27. The nutritional and/or therapeutic product according to claim 15 wherein a quantity of xenogeneic cells are also present, to create a hybrid product containing tissues from more than two animal species and further wherein at least some of said cells have been modified to express heterologous gene products.

28. The nutritional and/or therapeutic product according to claim 27 wherein said product contains cells other than muscle cells.

29. The nutritional and/or therapeutic product according to claim 28 wherein the cells are selected from the group consisting of myoblasts, osteoblasts, fibroblasts, lipoblasts, odontoblasts, adult neuronal progenitor cells, neural stem cells, multipotent stem cells from subventricular forebrain region, ependymal-derived neural stem cells, hematopoeitic stem cells, liver-derived hematopoeitic stem cells, marrow-derived stem cell, adipo-fibroblasts, adipose-derived stem cells, islet-producing stem cells, pancreatic-derived pluripotent islet-producing stem cells, mesenchymal stem cells, bone marrow stromal cells, muscle side population cells, bone marrow-derived recycling cells, blood-derived mesenchymal precursor cells, bone-marrow derived side population cells, muscle precursor cells, circulating skeleton stem cells, neural progenitor cells, multipotent adult progenitor cells, mesodermal progenitor cells, spinal cord progenitor cells and spore-like cells.

30. The nutritional and/or therapeutic product according to claim 22 wherein said mammalian cells are fat cells and further wherein said fat cells are incorporated into a topical cream.

31. The nutritional and/or therapeutic product according to claim 22 wherein said mammalian cells are include either hepatocytes and/or engineered liver tissue, and bile produced therefrom is incorporated into a nutritional supplement or oral or topical preparation.

32. The nutritional and/or therapeutic product according to claim 22 wherein said cells are tooth-derived cells or cells derived from tissue-engineered teeth or tusks.
